(19) United States
(12) Patent Application Publication
(10) Pub. No.: US 2017/0340808 A1
(43) Pub. Date: Nov. 30, 2017

(54) Kit Comprising a Medical Accessory Device and a Dummy Unit

(71) Applicant: Novo Nordisk A/S, Bagsvaerd (DK)

(72) Inventors: Jens Christian Andersen, Roskilde (DK); Nikolaj Frogner Krusell, Copenhagen OE (DK); Nikolaj Eusebius Jakobsen, Soeborg (DK)

(21) Appl. No.: 15/538,422

(22) PCT Filed: Jan. 14, 2016
(86) PCT No.: PCT/EP2016/050614

§ 371 Date: Jun. 21, 2017

(30) Foreign Application Priority Data
Jan. 16, 2015 (EP) 15151395.9

(51) Int. Cl. A61M 5/00; A61M 5/20; G09B 23/28; A61M 5/315; A61M 5/31

(52) U.S. Cl. CPC A61M 5/003; A61M 5/20; G09B 23/285; A61M 2005/3125; A61M 5/31568; A61M 2205/3327; A61M 2205/52; A61M 2005/3126

(57) ABSTRACT

The invention provides a kit comprising a medical accessory device and a dummy unit. The accessory device comprises first attachment means configured for releasable attachment of the accessory to co-operating second attachment means on an original device having an outer surface defining an original device space envelope. The dummy unit comprises a presentation surface on its external surface, a folded or rolled sheet, as well as dummy attachment means configured for releasable attachment of the accessory device to the dummy unit in co-operation with the first attachment means. By this arrangement the mounting of the accessory device on the original device can be demonstrated on the dummy unit in a simple and effective way.

The present invention relates to a kit comprising an accessory device and a dummy unit. In specific embodiments the invention relates to a packaging assembly providing user information in an intuitive way.

### BACKGROUND OF THE INVENTION

In the disclosure of the present invention reference is mostly made to the treatment of diabetes by subcutaneous drug delivery, either discrete or continuous, however, this is only an exemplary use of the present invention.

In relation to medical devices, more specifically accessory devices for medical devices it is crucial that the accessory device and the “main” device is used in the intended manner in order to avoid any risk of improper treatment and thereby potentially compromising the health of the patient. This is even more so the case with self-administered drug injection delivery devices, for example devices used to inject insulin in a patient's body.

Although a user, which may be the patient or a caregiver, may have received training in using the drug injection delivery device the user is often self-taught when it comes to the use of associated accessories.

Therefore it is of vital importance that the accessories are easy and intuitive to use but also that the accompanying instruction material is easy to understand such that they provide the patient with the ability to use an accessory as intended.

The quality of the accompanying instruction material and the abilities of the patient to comprehend the information in the instruction material determine the ease of which the accessories can be taken into use. Moreover, it is an important factor in reducing the rate of improper use and thereof following incorrect treatment. The accompanying instructions should therefore be developed towards users having most difficulty in understanding such information.

As an example, the accessory device may be adapted to create a log of the size and time of each drug injection, e.g. insulin injection. Addressing this problem a number of solutions have been proposed which would help a user to generate, collect and distribute data indicative of the use of a given medical device.

For example, WO 2007/107564 describes an electronic “add-on” module adapted to be attached to and measure signals generated by a standard mechanical pen device, the module relying on e.g. the sounds inherently produced by such a device during operation. Alternatively, in order to provide pre-filled drug delivery devices which more reliably allow detection of an out-dosed amount of drug, it has been proposed to modify such pre-filled drug delivery devices to provide them with structures making them more suitable for cooperation with external detection means, thereby providing more reliable and accurate determination of out-dosed drug amounts. For example, PCT/EP2012/069729 discloses a drug delivery device in which a rotating piston rod is provided with a magnet allowing an add-on logging module to detect the axial position of the magnet by means of 3D magnetometers. Further electronic add-on modules are disclosed in e.g. WO 99/02210 and WO 2013/120775.

Irrespective of the actual technology implemented in a given add-on logging module, it is important that such a module is introduced to a potential user in a way that will ensure that it is actually used and is also used correctly.

Having regard to the above, it is an object of the present invention to provide means for decreasing the risk of improper use of accessories for medical devices, especially devices intended for use by the patient unsupervised by a medical professional.

### DISCLOSURE OF THE INVENTION

In the disclosure of the present invention, embodiments and aspects will be described which will address the above object or which will address objects apparent from the below disclosure as well as from the description of exemplary embodiments.

Thus, in accordance with a general aspect of the invention a kit is provided comprising a medical accessory device and a dummy unit. The accessory device comprises first attachment means configured for releasable attachment of the accessory to co-operating second attachment means on an original device having an outer surface defining an original device space envelope. The dummy unit comprises a presentation surface on its external surface, a folded or rolled sheet, as well as dummy attachment means configured for releasable attachment of the accessory device to the dummy unit in co-operation with the first attachment means.

It is herewith achieved that the mounting of the accessory device on the original device can be demonstrated on the dummy unit. Based on the attachment of the accessory to the dummy unit the patient will better be able to mount the accessory correctly on the original device without other instructions than simply replacing the dummy unit with the original device.

As the dummy unit comprises a folded or rolled sheet the dummy unit can be made to resemble the shape and size of the original device, this providing the desired functionality in a simple and cost-effective way. The material for the sheet can for example be paper, carton, plastic or another suitable material that can be rolled or folded.

In the present application the term “space envelope” is a boundary description that defines shape of the space occupied by an object. This term is commonly used in the field of Computer-Aided-Design (CAD).

The original device is the device that the accessory is intended to be used with. Examples of original devices include medical devices, for example drug delivery injection devices.

The original device has a shape defined by its outer surface that defines its space envelope. The space envelope definition will be available to the skilled person designing the accessory device.

The accessory device may be in the form of an apparatus for detecting and logging the amount of drug administered and/or a timer alerting the patient of a pending injection.

The accessory device may comprise first attachment means that are used to releasable attachment of the accessory device to co-operating second attachment means on the original device, e.g. in the form of co-operating coupling structures.

In an exemplary embodiment the dummy unit is a non-working representation of the original device. At least part of the dummy unit is made to resemble the size and shape of the original part. Preferably the presentation surface has an image of the original unit applied to its presentation surface.

In an embodiment the presentation surface on the external surface of the dummy unit is designed to resemble the original device. The presentation surface could comprise 3D features for both tactile and visual resemblance and/or graphical features for visual resemblance.

The dummy unit may comprise instructions related to the use of the accessory.

In an exemplary embodiment the dummy attachment means has the same interface as the second attachment means on the original device, e.g. a friction fit. Therefore the interface between the first attachment means on the accessory and the dummy attachment means on the dummy unit functions in the same way as the interface between said first attachment means and the second attachment means on the accessory. Thus, the dummy unit can be used to simulate the mounting of the accessory on the original device.

An exemplary embodiment of the kit is further distinguished in that the external surface of the dummy unit defines a dummy unit space envelope, wherein at least a portion of the dummy unit space envelope is identical to or smaller than the original device space envelope. In this embodiment the dummy unit could be configured to occupy a space envelope bounded by an external surface that is substantially equal to the space envelope bounded by the outer surface of the original device. In an embodiment the dummy unit is configured for fitting inside the space envelope of the original device in the location of the second attachment means and the dummy attachment means respectively.

The dummy unit can have a high similarity to the original device. This improves the effectiveness of the dummy unit in providing a means for decreasing the risk of improper use of accessories for medical devices, especially devices intended for unsupervised use by the patient.

The folded or rolled sheet may be arranged to be unfolded or unrolled to reveal more information. For example, the dummy unit can be changed by unrolling or unfolding the paper sheet to display instructions to the user by printed information related to the use of the accessory on the reverse of the paper sheet which then becomes visible.

In an exemplary embodiment of the kit the dummy unit has a cross sectional shape selected among the following geometrical shapes: a circle, an oval, a stadium, a triangle, a square, a rectangle (e.g. a plane sheet of material), a pentagon, a hexagon, a heptagon, an octagon, a polygon or a combination of the aforementioned shapes. All of the shapes may be rolled and/or folded in paper or any other suitable sheet material. The dummy unit may be cylindrical with a first diameter and that the first attachment means comprises a body having a circular or C-shaped through-hole having a second diameter, wherein the second diameter is substantially equal to the first diameter, this allowing the accessory device to be mounted on the dummy unit by sliding onto the dummy unit. In an embodiment the diameter of the dummy unit is tapered from one end to the other. The first diameter is located between the two ends of the dummy unit allowing the accessory device to be press-fitted onto the dummy unit.

In a further exemplary embodiment the dummy unit has a tubular configuration having an internal free space, the kit further comprising at least one instruction unit, wherein the instruction unit comprises a folded or rolled sheet that is configured for fitting inside the internal free space of the dummy unit. It is herewith achieved that the instructions can be available with the dummy unit. In addition or alternatively, the dummy unit may on its presentation surface comprise information printed thereon representing the original device. It is herewith achieved that the dummy unit at least visibly will have the same appearance as the original device.

In an exemplary embodiment of the kit the original device is a drug delivery device, e.g. a drug injection device for injecting insulin. These devices are often self-administered by the patient and as such require clear instructions that are easy to understand.

In a further exemplary embodiment the kit comprises an accessory device in the form of a logging device comprising electronic circuitry adapted to create a log of expelled dose amounts of a drug. In this case the accessory could be designed to detect when it is removed from the dummy unit and placed on the original device e.g. by a mechanical switch. This will save the battery when the unit is in the package. Upon removal from the dummy unit the accessory could be activated. In a specific embodiment the kit further comprises an original device.

In accordance with a further aspect of the invention a kit is provided comprising a first unit adapted to be mounted to a second unit, the first unit having a first configuration and the second unit having a second configuration, the kit comprising a sheet folded or rolled into a presentation configuration corresponding to the second configuration.

The first unit may be an electronically controlled device having a feature set, the sheet comprises printed thereon information in respect of use of the first unit. The first unit may comprises a fully or partly ring-formed part, the sheet in its presentation configuration having been rolled into a generally cylindrical tube having a presentation surface, the ring-formed portion of the first unit being arranged around the tube.

The sheet may be provided with graphical information printed thereon corresponding to the presentation surface, the presentation surface depicting in the presentation configuration a drug delivery device. The depicted drug delivery device comprises one or more of a drug label, a dose setting window or display with a numeral, and dose setting means.

The first unit may be a logging device comprising electronic circuitry adapted to create a log of expelled dose amounts of drug.

The kit may be provided in combination with a drug delivery device having a configuration corresponding to the second configuration. The drug delivery device may comprise a drug reservoir or means for receiving a drug reservoir, and drug expelling means. The first unit is in the form of a logging device releasably attachable to the drug delivery device and comprising electronic circuitry adapted to create a log of expelled dose amounts of drug, comprising sensor means adapted to capture a property value related to a dose amount of drug expelled from a reservoir by the expelling means during an expelling event, and processor means adapted to determine dose amounts based on captured property values.

It should be emphasized that the term “comprises/comprising/comprised of” when used in this specification is taken to specify the presence of stated features, integers, steps or components but does not preclude the presence or addition of one or more other features, integers, steps, components or groups thereof.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides (e.g. insulins, insulin containing drugs, GLP-1 containing drugs as well as derivates thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs. Correspondingly, the term “subcutaneous” infusion is meant to encompass any method of transcutaneous delivery to a subject.

### BRIEF DESCRIPTION OF THE DRAWINGS

In the following embodiments of the invention will be described with reference to the drawings, wherein

FIG. 1 shows a perspective view of a medical accessory attached to an original device,

FIG. 2 shows a perspective view of a kit according to a first embodiment of the invention,

FIG. 3 shows a perspective view of the dummy unit and an instruction unit,

FIG. 4 shows a perspective view of the kit in FIG. 2 placed in its packaging, and

FIG. 5 shows a perspective view of kit according to a second embodiment of the invention.

In the following, the invention will be described in greater detail with reference to embodiments shown by the enclosed figures. It should be emphasized that the embodiments shown are used for example purposes only and should not be used to limit the scope of the invention.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part.

In the explanation of the figures, identical or corresponding elements will be provided with the same designations in different figures. Therefore, no explanation of all details will be given in connection with each single figure or embodiment.

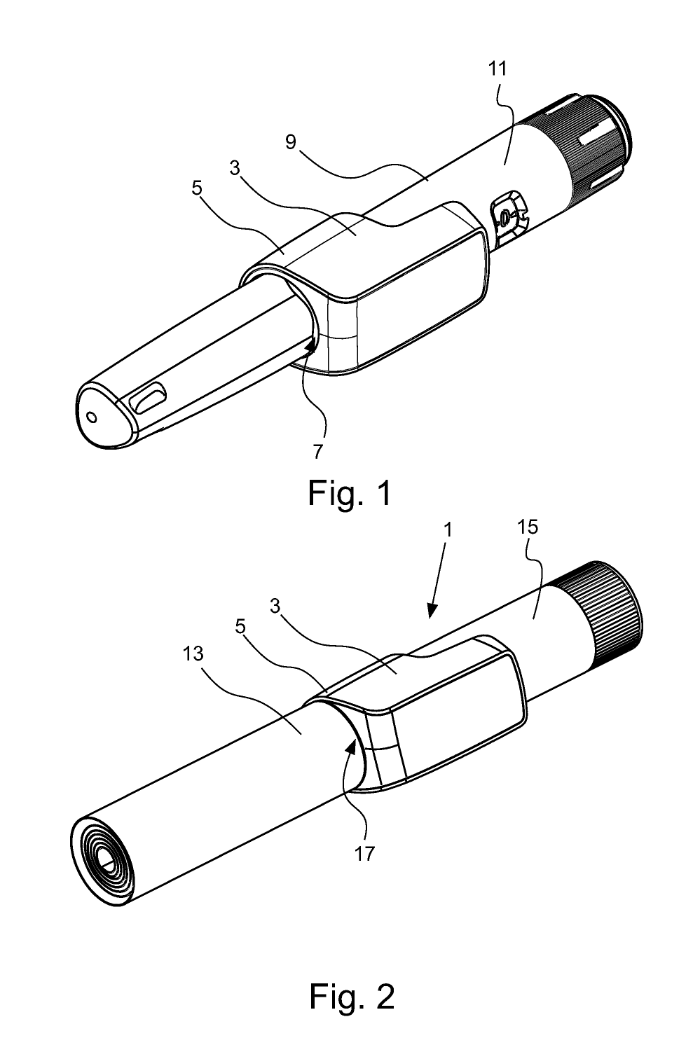

FIG. 1 shows a perspective view of a medical accessory device 3 attached to an original device 9. In the embodiment shown the original device is a generally pen-formed medical drug delivery device for a patient's self-administered injections of e.g. insulin. The original device has a space envelope bounded by an outer generally cylindrical surface 11.

The accessory device 3 has first attachment means 5 in the form of a circular through-hole formed in the body of the accessory device, the through-hole being provided with a first coupling structure. The first coupling structure co-operates with second attachment means 7 on the original device 9 in the form of corresponding second coupling means, this allowing the accessory device to be releasably mounted on the original device.

In the shown embodiment the medical accessory device is a logging device adapted to be releasably attached to a drug delivery device as shown. The drug delivery device comprises a drug reservoir, drug expelling means comprising dose setting means allowing a user to set a dose amount of drug to be expelled, a dose setting window or display with a numeral, and a drug label (not shown). A more detailed description of the logging device and the drug delivery as well as the principle of detection can be found in WO 2014/161952 which is hereby incorporated by reference.

Turning to FIG. 2, a perspective view of a kit 1 according to an aspect of the invention is shown, the kit comprising a medical accessory device 3 and a dummy unit 13. The medical accessory device is identical to the accessory device shown in FIG. 1. The dummy unit provides a representation of the original device 9 without having the same functionality.

The dummy unit 13 comprises a presentation surface 15 on its external surface. The presentation surface is provided with graphics that are configured for providing visual likeness with the original device. In the shown embodiment the presentation surface is printed in the same colour as the original drug delivery device and a dose setting member is printed on the proximal end if the presentation surface illustrating the rotatable dose setting member of the original drug delivery device. Additional characteristics such as a dose setting window and a drug label may be printed on the presentation surface.

The dummy unit 13 has dummy attachment means 17 in the form of a cylindrical portion with a first diameter. The first attachment means 5 in the form of the through-hole in the body of the accessory device 3 has a second diameter. The second diameter is substantially equal to the first diameter such that the first attachment means 5 and the dummy attachment means 17 releasably engage in a friction fit when the dummy unit 13 is inserted in the accessory device 3.

The first attachment means 5 in the form of the through-hole is thus able to interface with the second attachment means 7 and the dummy attachment means 17 in a similar way, albeit using different principles of engagement. By this arrangement it can be achieved that the placement of the accessory device 3 on the original device 9 can be demonstrated with the dummy unit 13.

In the embodiment shown in FIG. 2 the dummy unit 13 is made of a rolled sheet. The material for the sheet can be selected among paper, carton, cardboard, plastics, a combination of the aforementioned materials or another suitable material. The material shall be flexible enough to be rolled or folded into a shape that resembles the original device 9, just as it may be stiff enough to retain its shape in a no-load condition when rolled or folded.

The shown dummy unit may be cylindrical in shape or it may be rolled in a slightly conical form providing for ease mounting of the accessory device. The cross sectional shape of the dummy unit 13 in the embodiment shown is a circle.

In the embodiment shown the rolled sheet is in the form of an instruction manual for the accessory device and is arranged to be “unfolded” to reveal information. To avoid that the sheet will unroll when removed from the accessory device the sheet may be held in its tubular configuration by adhesive means which can then be released by the user.

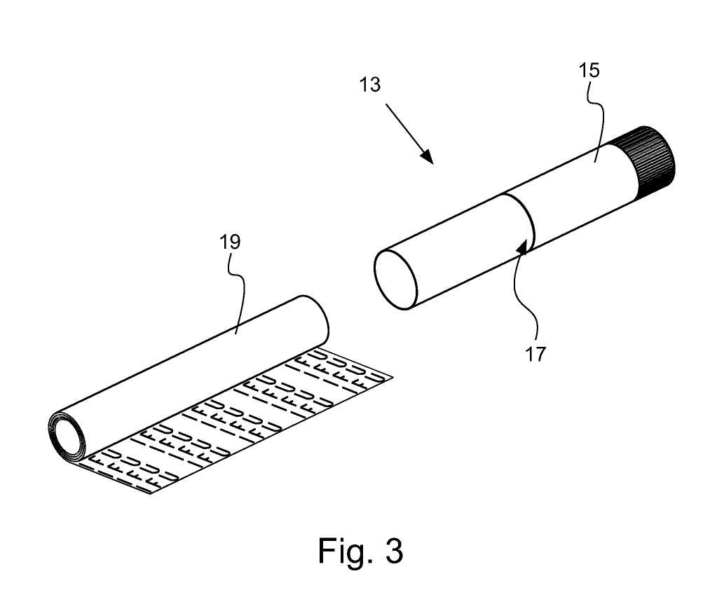

FIG. 3 shows a perspective view of a further dummy unit 13 and an instruction unit 19. The dummy unit 13 is in the form of a tube with an interior free space, the exterior surface of the dummy unit being provided with graphics that are configured for providing visual likeness with the original device 9. The instruction unit 19 is a sheet that is rolled into a tube that fits inside the internal free space of the dummy unit 13, the instruction unit 19 having information printed on it to assist the patient in using the accessory device 3.

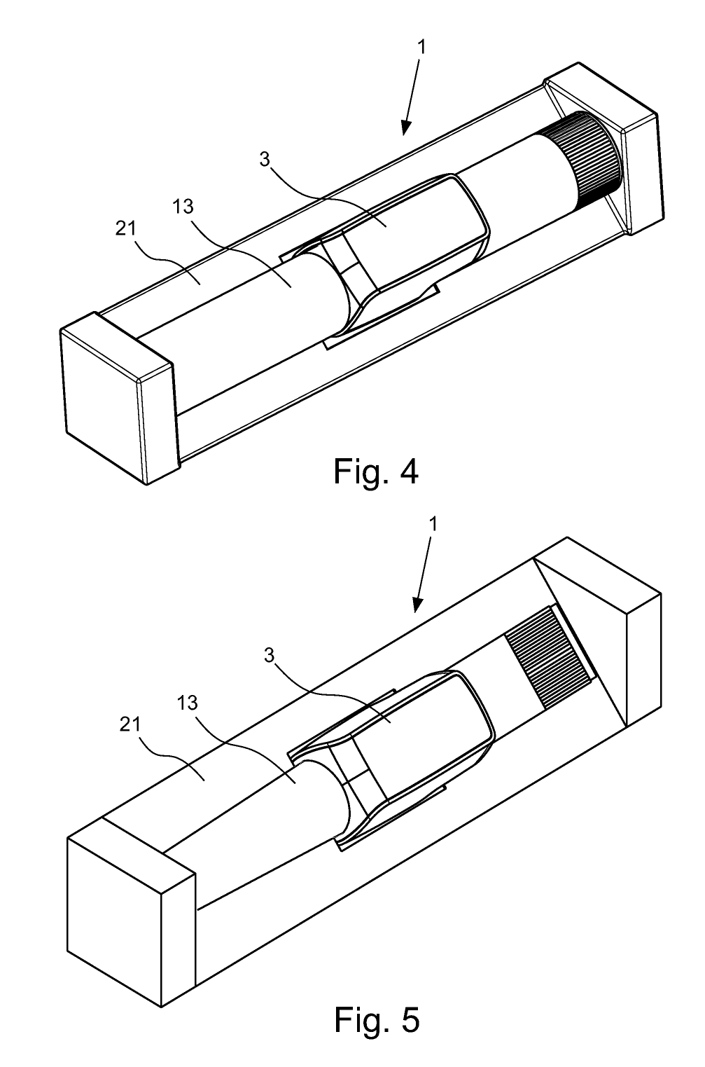

FIG. 4 shows a perspective view of the kit 1 in FIG. 2, wherein the kit 1 further comprises a packaging 21. This is how the patient may receive the accessory device 3. It is evident that the dummy unit 13 provides immediate and intuitive visual instructions to the patient as to how the accessory device 3 shall be mounted on an original device as shown in FIG. 1. As can be seen, the packaging comprises a pair of opposed end portions adapted to support the dummy unit as well as an inclined wall provided with an opening adapted to partially receive a mounted accessory device. The shown kit may be arranged in an outer box (not shown) which may be fully or partly transparent.

FIG. 5 shows a perspective view of a second embodiment of a kit 1 in accordance with an aspect of the invention, the kit 1 comprising a packaging 21. In this embodiment the dummy unit 13 is a flat sheet that can be folded from a larger sheet or made as part of the packaging, e.g. as an inclined wall as shown. The sheet is provided with an opening in which a “tongue” with a free end projects in the plane of the sheet, the tongue providing a dummy attachment means interfacing with the first attachment means 5 on the accessory device 3. This embodiment is a more simple way of providing visual and intuitive instructions to the patient. The instruction manual may be provided as a folded sheet arranged in the compartment formed behind the inclined wall.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A kit comprising;
a medical accessory device having first attachment structure configured for releasable attachment of the accessory device to co-operating second attachment structure on an original device having an outer surface defining an original device space envelope, and
a dummy unit comprising;
an external surface comprising a presentation surface,
a folded or rolled sheet, and
dummy attachment structure configured for releasable attachment of the accessory device to the dummy unit in co-operation with the first attachment structure.

2. A kit as in claim 1, wherein the external surface of the dummy unit defines a dummy unit space envelope, and wherein at least a portion of the dummy unit space envelope is identical to or smaller than the original device space envelope.

3. A kit as in claim 2, wherein said folded or rolled sheet is arranged to be unfolded or unrolled to reveal printed information.

4. A kit as in claim 1, wherein the dummy unit has a cross sectional shape selected among the following geometrical shapes: a circle, an oval, a stadium, a triangle, a square, a rectangle, a pentagon, a hexagon, a heptagon, an octagon, a polygon or a combination of the aforementioned shapes.

5. A kit as in claim 1, wherein the dummy unit has a cylindrical portion with a first diameter, the first attachment structure comprising a body having a circular or C-shaped through-hole having a second diameter, wherein the second diameter is substantially equal to the first diameter so that the accessory device can be attached to the cylindrical portion.

6. A kit as in claim 1, wherein the dummy unit is a tube having an internal free space, the kit further comprising at least one instruction unit, and wherein the instruction unit comprises a folded or rolled sheet that is configured for fitting inside the internal free space of the dummy unit.

7. A kit as in claim 1, wherein the dummy unit on its presentation surface comprises information printed thereon representing the original device.

8. A kit as in claim 1, wherein the original device is a drug delivery device.

9. A kit as in claim 1, wherein the accessory device is a logging device comprising electronic circuitry adapted to create a log of expelled dose amounts of a drug.

10. A kit as in claim 8, wherein the drug delivery device has an oblong generally pen-formed cylindrical configuration.

11. A kit as in claim 1, wherein the presentation surface comprises picture information printed thereon, the presentation surface depicting a drug delivery device comprising one or more of: a drug label, a dose setting window or display with a numeral, and dose setting structure.

12. A kit as in claim 1, wherein the kit further comprises an original device.
